(19) United States
(12) Patent Application Publication
(10) Pub. No.: US 2006/0223182 A1
(43) Pub. Date: Oct. 5, 2006

(54) Device for producing autologous VEGF

(76) Inventors: Thomas M. DiMauro, Southboro, MA (US); Mohamed Attawia, Canton, MA (US); Chantal Holy, Somerville, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/093,528

(22) Filed: Mar. 30, 2005

(51) Int. Cl. C12N 5/00; C12N 5/02

(52) U.S. Cl. 435/383; 435/395; 435/325

(57) ABSTRACT

A method of adding an effective amount of reactive oxygen species (ROS) to a mixture of macrophages to induce VEGF production by the macrophages. The induced macrophages are then added to a graft material.

### FIELD OF THE INVENTION

The present invention relates to biocompatible tissue implant devices for use in the repair of tissue injuries, organ disease states and/or congenital anomalies as well as methods for making and using such biocompatible tissue implant devices.

### BACKGROUND OF THE INVENTION

Injuries to tissue, such as cartilage, meniscus, intervertebral disc, skin, muscle, bone, tendon and ligament, where the tissue has been injured or traumatized, frequently require surgical intervention to repair the damage and facilitate healing. Such surgical repairs can include suturing or otherwise repairing the damaged tissue with known medical devices, augmenting the damaged tissue with other tissue, using an implant or a graft or any combination of these techniques.

One common tissue injury involves damage to cartilage, which is a non-vascular, resilient, flexible elastic connective tissue. Cartilage typically acts as a “shock-absorber” at articulating joints, but some types of cartilage provide support to tubular structures, such as for example, the larynx, air passages, and the ears. In general, cartilage tissue is comprised of cartilage cells, known as chondrocytes, located in an extracellular matrix, which contains collagen, a structural scaffold, and aggrecan, a space-filling proteoglycan. Several types of cartilage can be found in the body, including hyaline cartilage, fibrocartilage and elastic cartilage. Hyaline cartilage can appear in the body as distinct pieces, or alternatively, this type of cartilage can be found fused to the articular ends of bones. Hyaline cartilage is generally found in the body as articular cartilage, costal cartilage, and temporary cartilage (i.e., cartilage that is ultimately replaced by bone through the process of ossification). Fibrocartilage is a transitional tissue that is typically located between tendon and bone, bone and bone, and/or hyaline cartilage and hyaline cartilage. Elastic cartilage, which contains elastic fibers distributed throughout the extracellular matrix, is typically found in the epiglottis, the ears and the nose.

One common example of hyaline cartilage injury is a traumatic focal articular cartilage defect to the knee. A strong impact to the joint can result in the complete or partial removal of a cartilage fragment of variable size and shape. Damaged articular cartilage can severely restrict joint function, cause debilitating pain and may result in long term chronic diseases such as osteoarthritis, which gradually destroys the cartilage and underlying bone of the joint. Injuries to the articular cartilage tissue will not heal spontaneously and require surgical intervention if symptomatic. A modality of treatment consists of debridement and the removal of partially or completely unattached tissue fragments. In addition, the surgeon will often use a variety of methods such as abrasion, drilling or microfractures, to induce bleeding into the cartilage defect and formation of a clot. It is believed that the cells coming from the marrow will form a scar-like tissue, fibrocartilage, which can provide temporary relief to some symptoms. Unfortunately, fibrocartilage does not have the same mechanical properties as hyaline cartilage and degrades over time as a consequence of wear. Patients typically have to undergo repeated surgical procedures, which can lead to the complete deterioration of the cartilage surface. More recently, experimental approaches involving the implantation of autologous chondrocytes have been used with increasing frequency. The process involves the harvest of a small piece of articular cartilage in a first surgical procedure, which is then transported to a laboratory specialized in cell culture for amplification. The tissue piece is treated with enzymes that will release the chondrocytes from the matrix, and the isolated cells will be grown for a period of 3 to 4 weeks using standard tissue culture techniques. Once the cell population has reached a target number, the cells are sent back to the surgeon for implantation during a second surgical procedure. This manual, labor-intensive process is extremely costly and time consuming. Although the clinical data suggest long-term benefit for the patient, the prohibitive cost of the operation combined with the traumatic impact of two surgical procedures to the knee, have hampered adoption of this technique.

Therefore, there is a need for improved cartilage graft materials that can be quickly and easily produced.

In addition to the problems associated with cartilage repair, bone repair technology also has its unmet needs. For example, when an intervertebral disc degenerates significantly and causes pain, the degenerated disc is often removed and the vertebrae on each side of the disc are fused together via a bone graft interposed between them. However, despite sophisticated graft technology, spinal fusion does not routinely occur. It is hypothesized that angiogenesis is a key component of fusion, and that conventional fusion technologies do not adequately address the angiogenesis aspect of bone repair. Some conventional technologies use autograft as a way of enhancing fusion. Some use recombinant BMP. Some use stem cell concentrates. None of these technologies directly enhance the VEGF concentration of the graft.

Some technologies use PRP, which contains growth factors such as VEGF and TGF-β in concentrations of about 3-6 times that of whole blood.

To increase the chances of a successful fusion, the amount of VEGF (a known angiogenesis promoter) can be increased in the bone graft. VEGF-is particularly useful when the clinician uses large numbers of cells in the graft and so needs to ensure blood flow for adequate oxygen and glucose nutrition for those cells.

With the exception of PRP, none of the prior art enhancements provide any enhancement of VEGF. In general, whole blood has about 360 pg/ml of VEGF, while platelet concentrate provides VEGF in an amount of about 300% higher than whole blood.

### SUMMARY OF THE INVENTION

The present invention adds an effective amount of reactive oxygen species (ROS) to a mixture of macrophages to induce VEGF production by the macrophages. The induced macrophages are then added to a graft material.

Therefore, in accordance with the present invention, there is provided a method of enhancing tissue repair, comprising the steps of:
a) providing a mixture of macrophages,
b) contacting an effective amount of reactive oxygen species to the mixture to produce a formulation capable of inducing VEGF production in the macrophages, and
c) placing the formulation in a patient.

In some embodiments thereof, the inducer is a low concentration of reactive oxygen species (ROS). In other embodiments thereof, the reactive oxygen species (ROS) are produced via photocatalytic oxidation.

This invention may be carried out for soft tissue repair as well as bone fusion.

### DESCRIPTION OF THE FIGURES

FIG. 1 is a cross-section of a device of the present invention.

### DETAILED DESCRIPTION OF THE INVENTION

For the purposes of the present invention, “titanium dioxide” is also referred to as titania and TiO₂. A “UV light source” includes any light source emitting light having a maximum energy wavelength of between about 0.1 nm and about 380 nm. A “UVC light source” includes any light source emitting light having a maximum energy wavelength of between about 0.1 nm and less than 290 nm. A “UVB light source” includes any light source emitting light having a maximum energy wavelength of between 290 nm and less than 320 nm. A “UVA light source” includes any light source emitting light having a maximum energy wavelength of between 320 nm and less than 380 nm. A “visible light source” includes any light source emitting light having a maximum energy wavelength of between 380 nm and less than 780 nm. An “infrared light source” includes any light source emitting light having a maximum energy wavelength of between 780 nm and less than one million nm. A “reactive oxygen species” includes hydrogen peroxide, hydroxyl radicals, superoxide ion, and singlet oxygen and is also referred to as “ROS”.

A. Methods of Producing ROS

ROS can be added to the system in at least the following three ways:

i. Hydrogen Peroxide

In some embodiments, hydrogen peroxide (H₂O₂) is added in liquid form to the macrophage mixture in an amount sufficient to provide a final H₂O₂ concentration in the formulation of between 0.1 and 1 mM. Preferably, hydrogen peroxide (H₂O₂) is added to the macrophage concentrate in an amount sufficient to provide a final H₂O₂ concentration of between 0.25 and 0.75 mM.

ii. Superoxide Ion

In some embodiments, superoxide ion is added to the macrophage mixture in an amount sufficient to provide a final superoxide ion concentration in an amount sufficient to activate macrophages without causing cellular damage and DNS cross-linking.

iii. Photocatalytic Oxidation

The present inventors have developed a novel photocatalytic unit comprising a) a chamber for holding a macrophage concentrate, b) a photocatalytic layer and c) a light source. Upon photocatalytic illumination with light, the photocatalytic layer locally generates a plurality of reactive oxygen species, such as singlet oxygen. The reactive oxygen species (ROS) produced by this system are potent agents capable of providing the same oxidizing effect as H₂O₂.

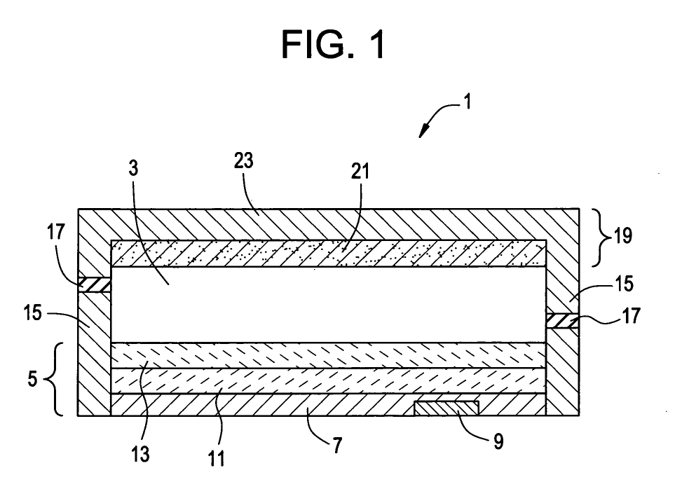

One potential photocatalytic device for producing autologous VEGF is shown in FIG. 1. There is provided a cross-section of a device 1 for producing autologous VEGF having a chamber 3, wherein the chamber defines:
a) a floor 5 comprising a lower layer UV light source 7 and a battery 9, an intermediate layer 11 of a UV-transmissable material (such as silica), and a photocatalytic upper layer 13 (such as TiO₂);
b) side walls 15 (preferably, light reflective) having ports 17 therein for the introduction and/or withdrawl of fluids from the chamber, and
c) an upper wall 19 comprising a lower layer 21 of a superinducer (such as cycloheximide) and a gas-impermeable upper layer 23.

The gas-impermeable cover prohibits oxygen from contacting fluids in the chamber.

In use, an aqueous solution comprising a concentrated amount of macrophages are injected into the chamber via one of the ports. The UV light source is then activated, thereby back-irradiating the titania and causing the TiO₂ to carry out a front-side photocatalytic reaction with the water in which it is in contact. The photocatalytic reaction not only produces ROS (thereby providing a VEGF-inducing agent), it also consumes oxygen (thereby promoting hypoxia). The clinician then waits a few hours to allow the ROS to induce the VEGF production from the macrophages and then withdraws the induced macrophages from the chamber via a port. The clinician may then mix the concentrated, induced macrophage mixture with the bone, nerve or soft tissue graft.

Therefore, in accordance with the present invention, there is provided a photocatalytic device for producing autologous VEGF, comprising:
a) a chamber adapted to retain a mixture comprising macrophages,
b) a photocatalytic layer within the chamber;
c) a light source adapted to irradiate the photocatalytic layer, and
d) an inducer capable of inducing VEGF production present within the chamber.

Preferably, the photocatalytic layer comprises a semiconductor material, and is preferably a metal oxide, more preferably titanium oxide. Titanium dioxide has been shown to have photocatalytic activity for generating ROS.

In some embodiments, the device is illuminated with an external source of light. For example, the light source could be the operating room lights. The light source could also be an external light box in which the device is placed just prior to implantation. In other embodiments, a fiber-optic wand is connected to an external light source and connected with the device to irradiate the titania.

In some embodiments, the photocatalytic layer is doped to enhance or prolong the photocatalytic effect. Some such dopants include, but are not limited to, metal alloys or ions of chromium and/or vanadium; phosphorescent compounds, ligands, or ions; organic compounds containing oxygen-rich chemical species such as peroxides, superoxides, acids, esters, ketones, aldehydes, ethers, epoxides, and lactones; and organic compounds containing conjugated systems, such as photostabilizers and dyes.

When the semiconductor element of the device of the present invention is properly irradiated by the UV light source, it is believed that reactive oxygen species (ROS) are produced at the semiconductor surface and enter the aqueous mixture adjacent the photocatalytic surface. These ROS include hydroxyl radicals (.OH), hydrogen peroxide (H₂O₂), superoxide ion (⁻O₂) and singlet oxygen (O). The photocatalytic oxidation (PCO) unit of the present invention can be tuned to emit ROS in both a magnitude and for a duration deemed appropriate for the extent of induction desired by the clinician.

When an effective amount of light irradiates the photocatalytic surface of the prosthetic device of the present invention, the sensitized surface can effectively catalyze both the oxidation of water (to produce hydroxyl radicals .OH) and the reduction of oxygen (to produce superoxide radicals ⁻O₂). Without wishing to be tied to a theory, it is believed that PCO may also produce significant amounts of hydrogen peroxide.

The PCO unit of the present invention comprises i) a light source and, ii) a photocatalytic surface comprising a semiconductor material to be irradiated by the light source. Without wishing to be tied to a theory, it is believed that, upon irradiation with an effective amount of UV light, the semiconductor material present in the photocatalytic surface produces holes and electrons. The holes catalyzes the oxidation of water, thereby producing hydroxyl radicals .OH. The electrons catalyze the reduction of oxygen, thereby producing superoxide radicals ⁻O₂.

Preferably, the semiconductor material comprises a solid catalyst comprising a transition element, and more preferably is selected from the group consisting of titanium dioxide and ferric oxide. More preferably, it comprises titanium dioxide. In some embodiments, the semiconductor is Degussa P25, available from DeGussa.

In some embodiments, the photocatalytic surface is produced by layering (preferably, by sonication) a powder comprising the semiconductor material upon a surface capable of being irradiated by the light source.

In some embodiments, there is provided a device of the present invention, wherein a photocatalytic layer is produced by oxidizing a base material comprising titanium, thereby producing a photocatalytic titania layer having a thickness (TH) of at least 0.2 μm.

Since photocatalysis is a surface phenomenon, the depth of the photocatalytic surface need not be particularly great. Moreover, it has been reported by Ohko, J. Biomed. Mat. Res. (Appl Biomat) 58: 97-101, 2001 that when TiO₂ thin films produced by heat treating exceed about 2 μm, the layer begins to peel from its substrate. Therefore, in some embodiments, the photocatalytic surface has a thickness of between about 0.2 μm and about 2 μm.

Preferably, the photocatalytic surface comprises a semiconductor material. More preferably, the semiconductor material is selected from the transition elements of the Periodic Table. More preferably, the semiconductor is selected from the group consisting of titanium dioxide and ferric oxide. More preferably, the semiconductor is titania. In some embodiments, the semiconductor is Degussa P25.

In some embodiments, the device comprises a chamber having a doped photocatalytic layer. In this case, the photocatalytic layer comprises a composite of a semiconductor material doped with a dopant that reduces the bandgap of the photocatalyst, thereby increasing the maximum wavelength of light absorbed by the photocatalytic layer. In some preferred embodiments, the dopant is selected from the group consisting of vanadium and chromium.

It has been reported by Anpo et al, Pure Appl. Chem. Vol. 72, (7), 2000, pp. 1265-70 that when a dopant selected from this group is ion-implanted onto a titanium dioxide surface, the resulting surface is substantially photocatalytically active when irradiated with white light.

In some other preferred embodiments, the dopant is nitrogen. It has been reported by Lin, J. Mater. Chem., 2003,13(12) 2996-3001 that when nitrogen is selected as the dopant, the resulting surface is substantially photocatalytically active when irradiated with light having either a 400 nm or a 550 nm wavelength.

In some other preferred embodiments, the dopant is selected from th group consisting of Nd⁺³, Pd⁺², Pt⁺⁴ and Fe⁺³. It has been reported by Shah, PNAS, Ap. 30, 2002, 99(S.2), pp. 6482-6 that when one of these dopants is selected is selected as the dopant, the resulting surface may be substantially photocatalytically active when irradiated with 450-460 nm light. Therefore, in some embodiments, the photocatalytic surface comprises a composite of a semiconductor material doped with a dopant that reduces the bandgap of the photocatalyst, thereby increasing the median wavelength of light absorbed by the photocatalytic layer to include wavelengths greater than UV.

In some preferred embodiments using a dopant, a titanium surface is oxidized to produce a titania surface layer, and this titania layer is then ion-bombarded with a dopant.

It is well known that there are many commercial Ti-based alloys commonly used in the medical devices that contain vanadium. One common example of such as alloy is Ti4Al6V alloy, which comprises 90 wt % titanium, about 4 wt % aluminum, and about 6 wt % vanadium. Without wishing to be tied to a theory, the present inventors believe that simple oxidation of this commercial alloy results in a photocatalytic layer comprising titania and vanadium. As noted above, this photocatalytic layer has special utility in that it can be activated by simple white light. The oxidized surface is a photocatalytic layer comprising titania and vanadium. In some embodiments, the photocatalytic layer activated by white light has a thickness of at least 1 um.

In some embodiments, the light transmissible material adjacent the photocatalytic layer is selected from the group consisting of a ceramic and a polymer. Suitable UV-transmissible ceramics include alumina, silica, CaF, titania and single crystal-sapphire. Suitable light transmissible polymers are preferably selected from the group consisting of polypropylene and polyesters.

In some embodiments, the light source is a UV light source. The UV light source is adapted to provide UV radiation to a UV-sensitive photocatalytic surface in an amount effective to produce an amount of ROS sufficient to induce VEGF production from the macrophage concentrate. In some embodiments, the UV light source has a spectral maximum in the range of the UV and near-UV components of the solar spectrum. Preferably, the light source has a spectral maximum in the range of the near-UV components of the solar spectrum. Preferably, the wavelength of the UV light is UVA light and emits light having a wavelength in the range of 320 and less than 380 nm. In some embodiments, the light source has a spectral maximum of about 356 nm. In this range, the UVA light effectively irradiates conventional TiO₂ and does not cause damage to DNA as does UVC light.

Preferably, when UV or near UV light sources are selected, they are used in conjunction with semiconductor materials that exhibit photocatalytic activity when irradiated by UV or near UV light. One preferred semiconductor suitably used with UV light is titania.

In other embodiments, the light source is a white light source. The white light source is adapted to provide white light to the photocatalytic surface in an amount effective to produce an effective amount of ROS. Preferably, the wavelength of the white light is in the range of 380 nm-780 nm. White light is particularly preferred because it effectively irradiates vanadium-doped TiO₂ or nitrogen-doped TiO₂ to produce photocatalysis and does not cause damage to DNA.

In some embodiments, using doped-titania as the photocatalytic surface, visible light having a maximum absorption wavelength of between 400 nm and 650 nm is used. In some embodiments, using doped titania as the photocatalytic surface, visible light having a maximum absorption wavelength of between 450 nm and 600 nm is used. In some embodiments, using doped titania as the photocatalytic surface, visible light having a maximum absorption wavelength of between 450 nm and 500 nm is used.

In some embodiments, the light source is situated to produce between about 0.1 watt and 100 watts of energy. Without wishing to be tied to a theory, it is believed that light transmission in this energy range will be sufficient to activate the photocatalytic surface. In some embodiments, the light source is situated to produce an energy intensity at the photocatalytic surface of between 0.1 watts/cm² and 10 watts/cm². In some embodiments, the light source is situated to produce about 1 milliwatt/cm². This latter value has been reported by Ohko et al., JBMR (Appl BioMat) 58: 97-101, 2001, to effectively irradiate a TiO₂ surface in an amount sufficient to produce a photocatalytic effect.

Since photocatalytic oxidation is generally believed to be a relatively ambient-temperature process, the heat produced by both the light source transmission and the desired oxidation reactions are believed to be negligible. That is, the temperature of the tissue surrounding the implant will not generally significantly increase during activation of the PCO unit, and so the viable cells will not be thermally harmed by the processes disclosed herein.

In some embodiments, ROS are added directly to the graft or to the PRP.

In some embodiments, the mixture containing the macrophages are aqueous.

B. Adjuncts

The amount of VEGF produced by contacting the macrophages with an effective amount of ROS may be enhanced by any of the following adjunct procedures:

i. Cycloheximide Superinduction

Brauchle, J. Biol. Chem., 271, 36, 21793-97, 1996, reports a strong superinduction effect provided by cycloheximide to human keratinocytes cultured in H₂O₂. The level of mRNA produced by the superinduction appear to be at least about 10 fold higher than that produced by the mere addition of H₂O₂. Therefore, in some embodiments, the chamber of the present invention includes a layer of cyclohexamide, preferably as an inner wall of the chamber. In others, cyclohexamine-coated beads are added to the chamber.

ii. Hypoxia

Ramanathan, Exp. Biol. Med., 228, 697-705, 2003, reports that culturing macrophages in a 1% O₂ environment increases VEGF production from about 50 pg/ml (normoxia) to about 300 pg/ml. Xiong., Am. J. Pathol., 1998, 153, 587-98 reports that that culturing RAW macrophages in an N₂/CO₂ environment increases the 48 hour VEGF production from about 1200 pg/ml (normoxia) to about 2000 pg/ml. Kuroki, J. Clin. Invest., 1996, 98, 1667-75 reports that hypoxia alone produces an increase in steady state VEGF mRNA levels in endothelial cells after 16 hours, while re-oxygenation produced an additional increase, with peak mRNA levels occurring 40-60 minutes after exposure to normoxia, thus paralleling the time course observed after exposure to exogenous ROS. Kuroki reported that ROS-associated VEGF mRNA increases were translated into VEGF protein when ROS were either directly added to cells, or when ROI were generated after reoxygenation of hypoxic cells. Kuroki identified ROS as the inducer of the reoxygenation response.

Therefore, in some embodiments, the amount of oxygen in the chamber is reduced prior to contacting the ROS to the macrophages.

iii. TGF-β

Stavri, Circulation, 1995, 92, 11-14 reports that exposing VSMCs to 2.5% O₂ for 4 hours causes a 1.5-2 fold increase in steady state levels of VEGF mRNA, and that a similar increase in expression was observed when the cells were incubated for the same length of time with 0.3 ng/ml TGF-β at 21% O₂. Stavri then reported that the combined treatment of TGF-β and incubation at 2.5% O2 caused a ˜7-fold increase in the level of VEGF mRNA, and concluded that this was greater than the additive effect of the two stimuli given alone.

It is noted that the 0.3 ng/ml TGF-β level may be achieved by the use of platelet releasate. Therefore, in one embodiment, PRP and hypoxia are used, and the resultant VEGF level could be at least 7-fold higher that of whole blood.

In some embodiments in which PRP is created by centrifugation of blood, a port may be created in a closed centrifugation container to provide a vacuum.

iv. TNF-α

Sen, J. Biol. Chem. 277, 36, 2002, 33284-90, reports that each of H₂O₂ (250 uM) and TNF-α (25 ng/ml) cause the VEGF production level in human keratinocytes to increasre from about 200 pg/ml to about 600 pg/ml. Sen further reports that simultaneous addition of the additives produced a VEGF production level of about 1200 pg/ml, and concluded that these effects were additive.

v. Priming

In many induction systems, it has been found that “priming” the environment with a small amount of the protein to be induced effects a large increase in the rate of protein production.

It is noted that PRP contains a significant amount of VEGF. Accordingly, in some embodiments, VEGF from PRP may be used as the priming agent.

In some embodiments, an anti-oxidant may be added to the graft after the ROS have activated the macrophages in order to attenuate any potential damage by the ROS.

C. Graft

In preferred embodiments, after the formulation containing macrophages has been induced to produce VEGF, the induced formulation is added to a graft material. Typically, the graft material may contain at least one of:

a) a carrier material,

b) viable cells, and

c) growth factors.

i. Carrier

When the graft material contains a carrier, the carrier is preferably adapted to remain within a graft site for a prolonged period and slowly release the VEGF contained therein to the surrounding environment. This mode of delivery allows the VEGF to remain in therapeutically effective amounts within the site for a prolonged period. By providing the VEGF within a carrier, the advantage of releasing the VEGF directly into the target area is realized.

In some embodiments, the carrier is provided in an injectable form. Injectability allows the carrier to be delivered in a minimally invasive, and preferably percutaneous method. In some embodiments, the injectable carrier is a gel. In others, the injectable carrier comprises hyaluronic acid (HA).

In some embodiments, the carrier of the graft comprises a porous matrix having an average pore size of at least 20 μm. Preferably, the porous matrix has an average pore size of between 20 μm and 100 μm. When the average pore size is in this range, it is believed that that porous matrix will also act as a scaffold for in-migrating cells capable of becoming cells of the desired tissue.

Examples of organic materials that can be used to form the porous matrix include, but are not limited to, collagen, polyamino acids, or gelatin.

The collagen source maybe allogenic, or xenogeneic relative to the mammal receiving the implants. The collagen may also be in the form of an atelopeptide or telopeptide collagen. Example of synthetic polymers that can be used to form the matrix include, but are not limited to, polylactic acids, polyglycolic acids, or combinations of polylactic/polyglycolic acids. Resorbable polymers, as well as non-resorbable polymers such as may constitute the matrix material. One of skill in the art will appreciate that the terms porous or semi-porous refers to the varying density of the pores in the matrix.

Preferred scaffolds for use in bone, cartilage, nerve, tendon, ligament, and meniscus repair include a gel that can be injected to the graft site, especially under arthroscopic fluid conditions. The gel can be a biological or synthetic gel formed from a bioresorbable or bioabsorbable material that has the ability to resorb in a timely fashion in the body environment.

Suitable scaffold agents include, but are not limited to, hyaluronic acid, fibrin glue, fibrin clot, collagen gel, alginate gel, gelatin-resorcin-formalin adhesive, mussel-based adhesive, dihydroxyphenylalanine-based adhesive, chitosan, transglutaminase, poly(amino acid)-based adhesive, cellulose-based adhesive, polysaccharide-based adhesive, synthetic acrylate-based adhesives, platelet rich plasma (PRP) gel, platelet poor plasma (PPP) gel, clot of PRP, clot of PPP, Matrigel, Monostearoyl Glycerol co-Succinate. (MGSA), Monostearoyl Glycerol co-Succinate/polyethylene glycol (MGSA/PEG) copolymers, laminin, elastin, proteoglycans, poly(N-isopropylacrylamide), poly(oxyalkylene), a copolymer of poly(ethylene oxide)-poly(propylene oxide), poly(vinyl alcohol) and combinations thereof.

Alternatively, the formulation can be mixed with a gel-like carrier prior to placement at the graft site.

In some embodiments, such as for meniscus repair, a pliable scaffold is preferred so as to allow the scaffold to adjust to the dimensions of the target site of implantation. For instance, the scaffold can comprise a gel-like material or an adhesive material, as well as a foam or mesh structure. Preferably, the scaffold can be a bioresorbable or bioabsorbable material. The scaffold can be formed from a polymeric foam component having pores with an open cell pore structure. The pore size can vary, but preferably, the pores are sized to allow tissue ingrowth. More preferably, the pore size is in the range of about 50 to 1000 microns, and even more preferably, in the range of about 50 to 500 microns. The polymeric foam component can, optionally, contain a reinforcing component, such as for example, woven, knitted, warped knitted (i.e., lace-like), non-woven, and braided structures. In some embodiments where the polymeric foam component contains a reinforcing component, the foam component can be integrated with the reinforcing component such that the pores of the foam component penetrate the mesh of the reinforcing component and interlock with the reinforcing component. In any of the above structures, mechanical properties of the material can be altered by changing the density or texture of the material, the type of knit or weave of the material, the thickness of the material, or by embedding particles in the material.

In some embodiments, the VEGF is predominantly released from a sustained delivery device by its diffusion through the sustained delivery device (preferably, though a polymer). In others, the VEGF is predominantly released from the sustained delivery device by the biodegradation of the sustained delivery device (preferably, biodegradation of a polymer).

Preferably, the carrier (i.e., sustained delivery device) comprises a bioresorbable material whose gradual erosion causes the gradual release of the VEGF to the graft site. In some embodiments, the carrier comprises a bioresorbable polymer. Preferably, the bioresorbable polymer has a half-life of at least one month, more preferably at least two months, more preferably at least 6 months such that new tissue generation coincides with scaffaold degradation.

In some embodiments, the carrier provides controlled release. In others, it provides continuous release. In others, it provides intermittent release. In others, the carrier comprises a biosensor.

In some embodiments, the sustained delivery device comprises the co-polymer poly-DL-lactide-co-glycolide (PLG).

In some embodiments, the sustained delivery device comprises a hydrogel. Hydrogels can also be used to deliver the VEGF in a time-release manner to the graft site. A “hydrogel” is a substance formed when an organic polymer (natural or synthetic) is set or solidified to create a three-dimensional open-lattice structure that entraps molecules of water or other solution to form a gel. The solidification can occur, e.g., by aggregation, coagulation, hydrophobic interactions, or cross-linking. The hydrogels employed in this invention rapidly solidify to keep the VEGF at the application site, thereby eliminating undesired migration from the gap. The hydrogels are also biocompatible, e.g., not toxic, to cells suspended in the hydrogel.

A “hydrogel-VEGF composition” is a suspension of a hydrogel containing desired VEGF. The hydrogel-VEGF composition forms a uniform distribution of VEGF with a well-defined and precisely controllable density. Moreover, the hydrogel can support very large densities of VEGF. In addition, the hydrogel allows diffusion of nutrients and waste products to, and away from, the site, which promotes tissue growth.

Hydrogels suitable for use in the present invention include water-containing gels, i.e., polymers characterized by hydrophilicity and insolubility in water. See, for instance, “Hydrogels”, pages 458-459, in Concise Encyclopedia of Polymer Science and Engineering, Eds. Mark et al., Wiley and Sons, (1990), the disclosure of which is incorporated herein entirely by reference. Although their use is optional in the present invention, the inclusion of hydrogels can be highly advantageous since they tend to contribute a number of desirable qualities. By virtue of their hydrophilic, water-containing nature, hydrogels can:

a) house viable cells, such as mesenchymal stems cells, and

b) assist with load bearing capabilities of the graft site.

In a preferred embodiment, the hydrogel is a fine, powdery synthetic hydrogel. Suitable hydrogels exhibit an optimal combination of such properties as compatibility with the matrix polymer of choice, and biocompatability. The hydrogel can include one or more of the following: polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers.

In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

ii. Cells

In most embodiments, viable cells from other tissues such as cells from the marrow can be combined with the induced macrophages to enhance repair. In one embodiment, healthy cells are introduced into the graft site that have the capability of at least partially repair the bone, ligament, cartilage, nerve or tendon. In some embodiments, these cells are introduced into the graft site and ultimately produce new extracellular matrix for the damaged tissue. Since it takes time for neighboring fibroblasts to migrate into the graft site, there will be a time advantage to providing a scaffold that already contains cells that are capable of producing the desired matrix.

In some embodiments, these cells are obtained from another human individual (allograft), while in others embodiments, the cells are obtained from the same individual (autograft). In some embodiments, the cells are taken from a similar tissue (e.g., they can be either bone, nerve, cartilage, tendon or ligament cells), while in others, the cells are taken from the non-graft site tissue (and may be mesenchymal stem cells). In others, autograft chondrocytes (such as from the knee, hip, shoulder, finger or ear) may be used.

In some embodiments, the viable cells are selected from the group consisting of osteoblasts, chondrocytes, heart cells, stem cells, olfactory ensheathing cells, and dopaminergic cells.

Preferably, when viable cells are selected as an additional therapeutic agent or substance, the viable cells comprise mesenchymal stem cells (MSCs). MSCs provide a special advantage for administration into a graft site because it is believed that they can more readily survive the environment present in the site; that they have a desirable level of plasticity; and that they have the ability to proliferate and differentiate into the desired cells.

In some embodiments, the mesenchymal stem cells are obtained from bone marrow, preferably autologous bone marrow. In others, the mesenchymal stem cells are obtained from adipose tissue, preferably autologous adipose tissue. In others, cell banks are used.

In some embodiments, the mesenchymal stem cells placed into the graft site are provided in an unconcentrated form. In others, they are provided in a concentrated form. When provided in concentrated form, they can be uncultured. Uncultured, concentrated MSCs can be readily obtained by centrifugation, filtration, or immuno-absorption. When filtration is selected, the methods disclosed in U.S. Pat. No. 6,049,026 (“Muschler”), the specification of which is incorporated by reference in its entirety, are preferably used. In some embodiments, the matrix used to filter and concentrate the MSCs is also administered into the graft site. If this matrix has suitable mechanical properties, it can be used to restore the length of the graft site that was lost during the degradation process. Preferably, the matrix comprises collagen.

iii. Growth Factors

As used herein, the term “growth factors” encompasses any cellular product that modulates the growth or differentiation of other cells, particularly connective tissue progenitor cells. The growth factors that may be used in accordance with the present invention include, but are not limited to, members of the fibroblast growth factor family, including acidic and basic fibroblast growth factor (FGF-1 and FGF-2) and FGF-4; members of the platelet-derived growth factor (PDGF) family, including PDGF-AB, PDGF-BB and PDGF-AA; EGFs; members of the insulin-like growth factor (IGF) family, including IGF-I and -II; the TGF-β superfamily, including TGF-β1, 2 and 3; osteoid-inducing factor (OIF), angiogenin(s); endothelins; hepatocyte growth factor and keratinocyte growth factor; members of the bone morphogenetic proteins (BMP's) BMP-1, BMP-3; BMP-2; OP-1; BMP-2A, BMP-2B, and BMP-7, BMP-14; HBGF-1 and HBGF-2; growth differentiation factors (GDF's) such as GDF-5 and rhGDF-5; members of the hedgehog family of proteins, including indian, sonic and desert hedgehog; ADMP-1; members of the interleukin (IL) family, including IL-1 thru IL-6; members of the colony-stimulating factor (CSF) family, including CSF-1, G-CSF, and GM-CSF; and isoforms thereof.

In some embodiments, the growth factor is administered in an amount effective to provide cell chemotaxis. This feature is useful when the clinician is relying upon cells adjacent the graft site to migrate into the graft site and begin producing ECM. Preferred chemotactic growth factors include BMPs and GDFs such as GDF-5 and rhGDF-5.

In some embodiments, the growth factor is administered in an amount effective to provide angiogenesis. This feature, which promotes, vascular development of the graft site, is useful when the clinician uses large numbers of cells and so needs to provide nutrition for those cells. Preferred angiogenetic growth factors include VEGF and EGF.

In preferred embodiments, a CDMP is administered in an amount effective to stimulate cell proliferation. This feature is useful when the clinician is uses moderately concentrated sources of cells (such as autologous point-of-care uncultured MSCs) to become a desired cell type. Preferred proliferation promoting growth factors include PDGF, TGF-β, IGF and BMPs. When stem cells are used, PDGF is a preferred proliferative growth factor.

In some embodiments, the growth factor is administered in an amount effective to stimulate cell differentiation. This feature is useful when the clinician is uses undifferentiated cells such as MSCs to become a desired cell type. Preferred differentiation growth factors include BMPs and GDFs such as GDF-5 and rhGDF-5.

In preferred embodiments, the growth factor is administered in an amount effective to stimulate extracellular matrix production. This feature is useful since such stimulation produces the ECM required for cellular ingrowth. Preferred ECM stimulating growth factors include GDFs such as GDF-5, TGF-P and IGF, and particularly rhGDF-5.

In some embodiments, platelet concentrate is provided as the effective growth factor. In one embodiment, the growth factors released by the platelets in the concentrate are present in an amount at least two-fold (e.g., four-fold) greater than the amount found in the blood from which the platelets were taken. In some embodiments, the platelet concentrate is autologous. In some embodiments, the platelet concentrate is platelet rich plasma (PRP). PRP is advantageous because it contains growth factors that can restimulate the growth of the ECM, and because its fibrin matrix provides a suitable scaffold for new tissue growth.

In addition, since PRP contains chemotactic growth factors, using PRP in combination with a GDF (preferably, rhGDF-5) provides a chemotactic carrier having a sustained release growth factor (rhGDF-5) having both mitogenic and ECM stimulating ability. This combination reduces the length of time over which rhGDF-5 needs to be released, while still providing chemotaxis, mitogenesis and ECM stimulation.

It has also been reported that exposing endothelial cells to ROS induces the production of VEGF therein. Therefore, in one embodiment, endothelial cells derived from bone marrow aspirate is contacted with ROS.

### EXAMPLE

This prophetic example describes a preferred embodiment of the present invention.

Blood is obtained from the patient and centrifuged to produce a buffy coat portion and a platelet button portion. The filtration and dewatering of blood are carried out in accordance with U.S. Pat. No. 5,733,545 (Hood), thereby providing a buffy coat having about 14×10⁶ monocytes/ml. These portions are separately removed from the remainder of the blood. The platelet button is then contacted with thrombin to cause the platelets to release growth factors and form a platelet releasate including TGF-β.

The platelet release and the concentrated buffy coat are then added to the device of FIG. 1 and irradiated with UV light to induce the production of VEGF in the macrophages.

Cho, Am. J. Physiol. Heart Circ. Physiol. 1280, 2001, H2357-63, reported upon that VEGF RNA and VEGF protein release are induced by hydrogen peroxide in human macrophages. In particular, Cho reported that exposing macrophages to a 0.5 mM concentration of H₂O₂ for about 30 minutes produces about 2.5 fg VEGF/cell. Inducing 1 ml of this monocyte-rich buffy coat with ROS should produce about 2 ml of 17 ng/ml VEGF (based upon Cho's production rate of 2.5 fg VEGF/cell).

The 17 ng/ml VEGF concentration in this mixture is about 50 times that of whole blood. Since this formulation has also been contacted with TGF-β, it is expected that the final VEGF production should be even greater.

We claim:

1. A method of enhancing tissue repair, comprising the steps of:
a) providing a mixture of macrophages,
b) contacting an effective amount of reactive oxygen species to the mixture to produce a formulation capable of inducing VEGF production in the macrophages, and
c) placing the formulation in a patient.

2. The method of claim 1 wherein the mixture comprises concentrated macrophages.

3. The method of claim 1 wherein the reactive oxygen species are contacted to the mixture by adding a solution comprising an effective amount of reactive oxygen species to the mixtures.

4. The method of claim 3 wherein the solution comprises an effective amount of hydrogen peroxide.

5. The method of claim 1 further comprising the step of:
d) administering an anti-oxidant to the patient.

6. The method of claim 1 wherein the reactive oxygen species are contacted to the mixture by contacting a photocatalytic material to the mixture and irradiating the photocatalytic material.

7. The method of claim 6 wherein the photocatalytic material comprises a metal oxide.

8. The method of claim 7 wherein the metal oxide is a semiconductor oxide.

9. The method of claim 8 wherein the semiconductor oxide is titania.

10. The method of claim 9 wherein the photocatalytic material is doped.

11. The method of claim 6 wherein the irradiation is accomplished with UV light.

12. The method of claim 6 wherein the irradiation is accomplished with white light.

13. A photocatalytic device for producing autologous VEGF, comprising:
a. a chamber adapted to retain a mixture comprising macrophages,
b. a photocatalytic layer within the chamber;
c. a light source adapted to irradiate the photocatalytic layer, and
d. an inducer capable of inducing VEGF production present within the chamber.

14. The device of claim 13 wherein the photocatalytic layer forms a first wall in the chamber.

15. The device of claim 13 wherein the photocatalytic layer is present as beads within the chamber.

16. The device of claim 13 wherein the chamber comprises a port for the introduction and withdrawl of the mixture from the chamber.

17. The device of claim 13 wherein the chamber comprises a light transmissible material.

18. The device of claim 13 wherein the chamber comprises a UV transmissible material.

19. The device of claim 13 wherein the light source is a UV light source.

20. The device of claim 13 wherein the inducer comprises cyclohexamide.

21. A graft for tissue repair, comprising:
a) viable cells, and
b) macrophages induced to produce VEGF.

22. The graft of claim 21 wherein the viable cells are selected from the group consisting of osteoblasts and chondrocytes.

23. The graft of claim 21 wherein the viable cells comprise heart cells.

24. The graft of claim 21 wherein the viable cells comprise stem cells.

25. The graft of claim 21 wherein the viable cells comprise olfactory ensheathing cells.

26. The graft of claim 21 wherein the viable cells comprise dopeminergic cells.

27. The graft of claim 21 wherein the macrophages are present in a concentration of at least 10⁶ cells/cc.

28. The graft of claim 21 wherein the macrophages are present in a concentration of at least 5×10⁶ cells/cc.

29. The graft of claim 21 further comprising:
c) a carrier.

30. The graft of claim 29 wherein the carrier is injectable.

31. The graft of claim 21 further comprising:
c) a second growth factor.

32. The graft of claim 31 wherein the second growth factor is GDF-5.

33. The graft of claim 31 wherein the second growth factor is rhGDF-5.

34. The graft of claim 31 wherein the second growth factor is derived from PRP.

35. The graft of claim 31 wherein the second growth factor is TGF-β.

36. A method of enhancing tissue repair, comprising the steps of:
a) providing a mixture of endothelial cells,
b) contacting an effective amount of reactive oxygen species to the mixture to produce a formulation capable of inducing VEGF production in the macrophages, and
c) placing the formulation in a patient.
